(19) United States
(12) Patent Application Publication
(10) Pub. No.: US 2026/0216470 A1
(43) Pub. Date: Jul. 30, 2026

(54) METHOD, SYSTEM AND NON-TRANSITORY COMPUTER-READABLE MEDIUM FOR PRODUCING NEUROMODULATING AUDIO SIGNALS FOR ALTERING ENDOGENOUS ELECTRICAL SIGNALS

(71) Applicant: Elemind Technologies, Inc., Cambridge, MA (US)

(72) Inventors: Scott Clarke BRESSLER, Scituate, MA (US); Meredith PERRY, Cambridge, MA (US); Heather Lynn READ, Storrs Mansfield, CT (US); David Cheng-Ping WANG, Cambridge, MA (US)

(21) Appl. No.: 19/143,702

(22) PCT Filed: Dec. 28, 2023
(86) PCT No.: PCT/US2023/086238

§ 371 Date: Jun. 26, 2025

Related U.S. Application Data
(60) Provisional application No. 63/477,965, filed on Dec. 30, 2022.

(51) Int. Cl. A61M 21/02; A61M 21/00

(52) U.S. Cl. CPC A61M 21/02; A61M 2021/0027

(57) ABSTRACT

Provided herein are systems, devices, and methods for acoustic neuromodulation of a user using audio signals. The method can include measuring an endogenous electrical signal of a user, presenting a background audio signal to the user, generating a neuromodulating audio signal based on the measured endogenous electrical signal, the neuromodulating audio signal configured to alter at least one characteristic of the endogenous electrical signal, and intermittently presenting the neuromodulating audio signal to the user to alter the at least one characteristic of the endogenous electrical signal. The at least one characteristic of the endogenous electrical signal can include a wavelength, phase, or amplitude of the endogenous electrical signal.

### CROSS REFERENCE TO RELATED APPLICATIONS

This application claims priority to and the benefit of U.S. Provisional Patent Application Ser. No. 63/477,965, filed Dec. 30, 2022, the entire contents of which are hereby incorporated by reference.

### BACKGROUND

Neuromodulation includes inhibition, stimulation, modification, regulation, or therapeutic alteration (e.g., “modulation”) of nervous system function. Electrical or chemical agents can be delivered to a target area to modulate the nerve activity. Auditory signals, such as pink noise or white noise, have also been found to be useful neuromodulators for some applications, such as for improving sleep. However, the auditory signals conventionally utilized for neuromodulation can be unpleasant for the user to listen to. Accordingly, there remains a need for alternative methods of producing neuromodulating audio signals.

### SUMMARY

Described herein are systems, devices, and methods for acoustic neuromodulation of an endogenous electrical signal using a masked audio signal. The methods provided herein can include generating and presenting a non-neuromodulating audio signal, generating a neuromodulating audio signal based on a measured endogenous electrical signal of a user, and intermittently presenting the neuromodulating audio signal to alter the endogenous electrical signal of the user. For example, the neuromodulating audio signal can alter a wavelength, phase, and/or amplitude of the endogenous electrical signal. The neuromodulating audio signal may be presented simultaneously or near-simultaneously with the continuously presented non-neuromodulating audio signal. In this manner, the neuromodulating audio signal can be masked, or hidden, by the non-neuromodulating audio signal. The non-neuromodulating audio signal can include a pleasant soundtrack that can improve the neuromodulation experience of the user. In some instances, the neuromodulating audio signal can also include a pleasant soundtrack.

In some examples, a method for acoustic neuromodulation is provided, comprising: measuring an endogenous electrical signal of a user; presenting a background audio signal to the user; based on the measured endogenous electrical signal, generating a neuromodulating audio signal configured to alter the endogenous electrical signal; and intermittently presenting the neuromodulating audio signal to the user to alter at least one characteristic of the endogenous electrical signal.

In some examples, a system for acoustic neuromodulation is provided, comprising: one or more sensors configured to measure an endogenous electrical signal of a user; a processing device configured to: generate a background audio signal; and based on the measured endogenous electrical signal, generate a neuromodulating audio signal configured to alter at least one characteristic of the endogenous electrical signal; and one or more speakers configured to: present the background audio signal to the user; and intermittently present the neuromodulating audio signal to the user to alter the at least one characteristic of the endogenous electrical signal.

In some examples, a non-transitory computer-readable storage medium is provided, the computer-readable storage medium storing instructions, that, when executed by a processing device operatively coupled to one or more sensors configured to measure an endogenous electrical signal of a user and to one or more speakers configured to present audio signals, cause the device to: generate a background audio signal; based on the measured endogenous electrical signal, generate a neuromodulating audio signal configured to alter at least one characteristic of the endogenous electrical signal; and cause the one or more speakers to: present the background audio signal to the user; and intermittently present the neuromodulating audio signal to the user to alter the at least one characteristic of the endogenous electrical signal.

### BRIEF DESCRIPTION OF THE FIGURES

Various aspects of the disclosed systems and methods are set forth with particularity in the appended claims. A better understanding of the features and advantages of the disclosed systems and methods will be obtained by reference to the detailed description of illustrative embodiments and the accompanying drawings.

FIG. 1 illustrates exemplary phases of a brainwave of a user and a neuromodulating (e.g., stimulating) signal that are phase-locked with one another, in accordance with some embodiments.

FIG. 2 illustrates a system architecture of an exemplary acoustic neuromodulation device, in accordance with some embodiments.

FIG. 3 illustrates an exemplary method for altering an endogenous electrical signal of a user using a neuromodulating audio signal, in accordance with some embodiments.

FIG. 4 illustrates an exemplary method for altering an endogenous electrical signal of a user using rain drop sounds, in accordance with some embodiments.

FIG. 5 illustrates a computing device, in accordance with some embodiments.

### DETAILED DESCRIPTION

Described herein are systems, devices, and methods for generating a neuromodulating audio signal that is configured to alter an endogenous electrical signal of a user and presenting the neuromodulating audio signal with another audio signal that does not significantly alter the endogenous electrical signal of the user. Accordingly, the neuromodulating audio signal can be “masked” by or hidden within another, preferably more pleasant, soundtrack, thereby improving the experience for the user. As used herein, neuromodulation includes (without limitation) inhibition, stimulation, modification, regulation, or therapeutic alteration (e.g., “modulation”) of nervous system function and the neuromodulating audio signal inhibits, stimulates, modifies, regulates, or therapeutically alters (e.g., “modulates”) nervous system function. In some instances, the masking audio signal is presented continuously to the user and the neuromodulating audio signal is presented intermittently to the user while in use.

Neuromodulating audio signals can be used, for example, to modulate a user's physiological, perceptual, cognitive, and/or behavioral states. More particularly, the neuromodulating audio signal can be configured to alter a characteristic (e.g., wavelength, phase, amplitude, etc.) of an endogenous electrical signal of a user (e.g., an EEG signal). The predefined state of the user (e.g., the effect of the altered endogenous electrical signal) can be determined or set based on a time of day, a state or condition of the user's environment, the user's actions, policies, expression of an individual's desires, habits, tendencies, or behaviors, or a combination of any of these. The predefined state of the user can be, for example, a final behavior state or an intermediate behavior state at any point or period in time defined by a user, individual, or policy.

In some examples, the state of the user (both the current state of the user and the predefined state of the user) can be associated with the endogenous electrical signal of the user. The endogenous electrical signal of the user is an electrical signal that is produced by neural tissue of the user, which can include, for example, brain signals and/or brain waves. The endogenous electrical signal varies over time and can correspond to different brain states of a subject. For example, in a first mental state, the user may exhibit a first set of endogenous electrical signals with a first set of characteristics, and in a second mental state, the user may exhibit a second set of endogenous electrical signals with a second set of characteristics. The characteristics associated with the endogenous electrical signals can include, for example, a wavelength, a frequency, an amplitude, a phase, a center frequency of a frequency band, a phase difference, a variance, a co-variance, a spindle, a K-complex, or any other physical property associated with the endogenous electrical signal.

The endogenous electrical signal of the user can be measured using one or more sensors, which can be in the form of electrode(s) such as an implanted electrode, a surface electrode array, an encephalogram (EEG) electrode, or any other type of electrode that can be attached to or placed in contact with a portion of the user's body (e.g., an ear or a forehead of the user). In some examples, the electrode can neuromodulate brain tissue, record neural activity, or both. The electrode can be used alone or with one or more external recording electrodes.

As described herein, a neuromodulating audio signal can be intermittently presented to the user to alter the endogenous electrical signal of the user.

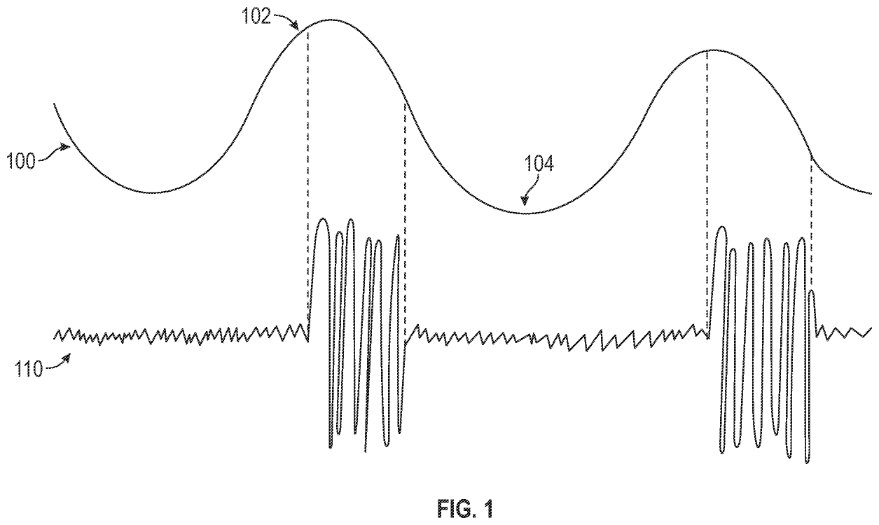

In some examples, the neuromodulating audio signal can be phase-locked with a detected instantaneous phase of one or more endogenous electrical signals. As used herein, to say that a first signal is “phase-locked” with a second signal means that an alteration of the first signal occurs relative to the phase of the second signal. In particular, brain waves occur at frequencies below auditory frequencies, so if an audio signal oscillates in the same phase as a brain wave, an individual wouldn't hear it. Accordingly, to provide an auditory signal that is perceptible to an individual, the phase of the two signals may have a fixed-offset, rather than equal phase. FIG. 1 illustrates an example brain wave signal 100 and an audio signal 110 phase-locked to the brain wave signal 100 at a fixed-offset. In some embodiments, the first signal (e.g., audio signal 110) can be presented when the second signal (e.g., brain wave signal 100) reaches a “peak” phase 102. In some embodiments, the first signal can be presented intermittently, i.e., when two “peaks” of the second signal are skipped. In other words, the first signal may not presented during these skipped peaks of the second signal. In this example, the first signal can be presented again at the fourth peak phase of the second signal. Other presentation schemes of the first signal relative to the second signal are possible and contemplated. In some examples, the neuromodulating audio signal can be pulsed, where pulses of the neuromodulation each occur at or shortly before a peak of an endogenous wave (e.g., a theta wave, an alpha wave, a delta wave, or any other type of neural oscillation that originates from a user's brain). As another example, pulses of the neuromodulation can each occur at or shortly before a trough phase 104 of the endogenous wave. In some examples, pulses of the neuromodulation can each occur at about 90 degrees (e.g., at, slightly more than, or slightly less than 90 degrees) before a peak or a trough of the endogenous wave. Accordingly, the neuromodulating audio signal can be presented intermittently to the user to alter a characteristic (e.g., the wavelength, phase, or amplitude) of the endogenous electrical signal of the user.

In some examples, the neuromodulating audio signal is generated based at least in part on another audio signal that does not significantly alter a characteristic (e.g., a wavelength, phase, and/or amplitude) of the endogenous electrical signal of the user. This other audio signal may otherwise be referred to herein as the non-neuromodulating, or background, audio signal. The background audio signal can be presented agnostic to characteristics (e.g., phase) of the endogenous electrical signal. Stated otherwise, the background audio signal can be independent of the endogenous electrical signal. In contrast, the neuromodulating audio signal can have a consistent (e.g., phase-locked) relationship with the endogenous electrical signal to produce a neuromodulating effect in the endogenous electrical signal. The neuromodulating audio signal can be generated to complement the non-neuromodulating audio signal. In some examples, the neuromodulating audio signal is produced by modifying the non-neuromodulating audio signal. For example, one or more characteristics, or acoustic features, of the non-neuromodulating audio signal can be altered to produce the neuromodulating audio signal. In some embodiments, the non-neuromodulating audio signal can be presented continuously to the user and the neuromodulating audio signal can be presented intermittently to the user.

In some embodiments, the neuromodulating audio signal can be presented with the non-neuromodulating audio signal so as to “mask,” blend, or hide the neuromodulating audio signal within the non-neuromodulating audio signal, which may be effective to improve the user experience. More particularly, intermittent presentation of the neuromodulating audio signal along with an ongoing presentation of the non-neuromodulating audio signal can make the neuromodulating audio signal less jarring or even pleasing to the user. In such embodiments, the non-neuromodulating audio signal can be continuously presented to the user with the neuromodulating audio signal being presented intermittently to the user. It should be appreciated that the time between intermittent presentations of the neuromodulating audio signal can be based on the measured endogenous electrical signal of the user, so as to phase-lock the neuromodulating audio signal with the endogenous electrical signal of the user, as described above.

As described herein, the neuromodulating audio signal and the audio signal that does not significantly alter the endogenous electrical signal of the user can each be an audio signal that represents, mimics, or approximates a sound-producing event. Sound-producing events can include events occurring in nature as well as events that do not occur in nature. Sound-producing events can additionally or alternatively include synthesized sounds that are not readily identifiable by the user. By way of example and not limitation, sound-producing events can include rain drop sounds (e.g., rain drops on various surfaces, rain drops into a puddle, rain drops of various intensities), thunderstorm sounds (e.g., rain, thunder, wind, etc.), sounds of a flow of water (e.g., dripping water, a steady stream of water, a river flowing, a waterfall, etc.), sounds of a bubbling brook, ocean sounds (e.g., waves crashing, seagulls, boat horns blasting, whales calling, wildlife splashing or jumping, etc.), forest sounds, leaf sounds, wildlife sounds (e.g., birds chirping, frogs croaking, crickets chirping, etc.), record player sounds (e.g., pops and clicks sometimes referred to as surface noise, needle chatter, etc.), sounds of children playing (e.g., squeaks of playground equipment, children calling and laughing, chains of a swing clanking, etc.), logs crackling in a fire, traffic sounds (e.g., horns honking, engines idling, rolling tires on pavement, etc.), synthesized sounds (e.g., pure tones, pure tone modulated by another tone, binaural beats, etc.) and variations and combinations thereof.

In some embodiments, the neuromodulating audio signal and the non-neuromodulating audio signal can include sounds simulating the same type of sound-producing event. For example, the non-neuromodulating audio signal can simulate a rainstorm, and the neuromodulating audio signal can simulate larger, or “fatter”, rain drops within the rainstorm. As another example, the non-neuromodulating audio signal can simulate a bubbling brook, and the neuromodulating audio signal can simulate different sounds of a bubbling brook, such as a particularly large bubble in the brook or something falling into the brook. In still another example, the non-neuromodulating audio signal can simulate the sounds of a forest, and the neuromodulating audio signal can simulate different forest sounds, such as a bird chirping in the forest. In some examples, the neuromodulating audio signal generates sounds that have a salient perceptual difference from sounds generated by the non-neuromodulating audio signal. In general, a sound that has a salient perceptual difference from another sound is a sound to which the brain responds differently (e.g., the brain identifies the sound as being unusual or different or the sound evokes an auditory response in the brain).

In some embodiments, the neuromodulating audio signal and the audio signal that does not significantly alter the endogenous electrical signal of the user are provided at different sound pressure levels (SPL) calculated from the root mean square (RMS) energy of each of the signals. To ensure an effective signal-to-noise ratio, the neuromodulating audio signal can be presented at an SPL that is at least 4 dB greater than the SPL of the audio signal that does not significantly alter the endogenous electrical signal of the user. For example, the SPL of the neuromodulating audio signal can be at least 4 dB, at least 5 dB, at least 10 dB, at least 15 dB, at least 20 dB or even higher than the SPL of the audio signal that does not significantly alter the endogenous electrical signal of the user. In some examples, the SPL of the non-neuromodulating audio signal is from about 23 dB to about 60 dB, from about 23 dB to about 50 dB, from about 30 dB to about 60 dB, from about 30 dB to about 50 dB, from about 40 dB to about 60 dB, or from about 40 dB to about 60 dB, including any and all ranges and sub-ranges therein. In some examples, the SPL of the neuromodulating signal is from about 50 dB to about 85 dB, from about 50 dB to about 75 dB, from about 60 dB to about 85 dB, or from about 60 dB to about 75 dB, including any and all ranges and sub-ranges therein.

In some embodiments, the neuromodulating audio signal is presented as a pulse having a duration of at least 5 ms, at least 10 ms, at least 15 ms, at least 20 ms, or at least 25 ms. In some embodiments, the duration of the pulse is less than about 50% of the period of the brainwave to which the neuromodulating audio signal is phase-locked. For example, the duration of the pulse of the neuromodulating audio signal can be about 10 ms, about 25 ms, about 50 ms, or greater, depending on the particular implementation.

In some embodiments, the audio signals (e.g., the neuromodulating audio signal and the audio signal that does not significantly alter the endogenous electrical signal of the user) are output from an output device. The output device can include, by way of example and not limitation, an audio speaker or other audio source configured to transmit or emit audio signals.

The output device and the electrode for measuring the endogenous electrical signal of the user can be incorporated into one or more attachment devices for securing or coupling the electrode to the user's body to enable the electrode to detect the user's endogenous electrical signals. Attachment devices can include, for example, a headband, an adjustable strap, etc. In some embodiments, the attachment device is a headband. The headband can further include a central processing unit (CPU), a power source (e.g., a battery), and one or more additional sensors. When included, one or more additional sensors can be used to obtain additional data pertaining to a physiological or physical condition of the user. Additional sensors can include, for example, oximeters, light sensors, optical sensors, temperature sensors, motion sensors, humidity sensors, microphones, electromyograms, electrooculograms, electrocardiograms, etc. In some embodiments, the electrode and one or more additional sensors are incorporated into a sensing module. In some embodiments, the electrodes, sensors, CPU, and power source may be packaged together into a single housing or device. In alternative embodiments, it is contemplated that the components of the system can be housed in two or more devices that are communicatively coupled to one another. For example, the sensors can be incorporated into a tabletop device that includes one or more plug-in, wired EEG electrodes. The sensors, including the EEG electrodes, can collect data and transmit the data to an external device (e.g., a desktop computer, a laptop computer, or another remotely-located computing device) for processing. As another example, the sensors can be incorporated into a wearable device that is configured to communicate with a tabletop device that conducts the processing.

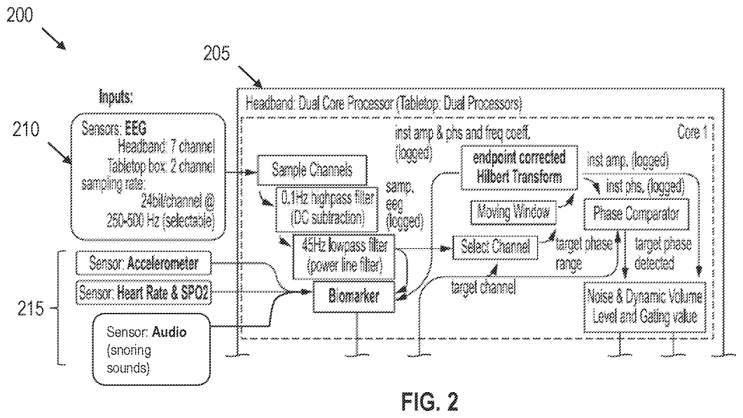

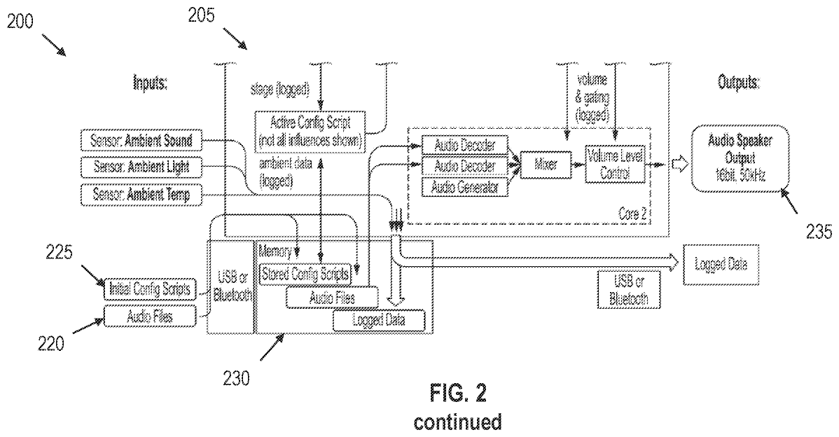

FIG. 2 schematically illustrates an example software architecture of an acoustic neuromodulation system 200. The system 200 can be used for generating or modifying a neuromodulating audio signal that is configured to alter an endogenous electrical signal of a user, and presenting the neuromodulating audio signal with another audio signal that does not significantly alter the endogenous electrical signal of the user. In some embodiments, the system 200 can be configured to record EEG and compute biomarkers. As shown in FIG. 2, a plurality of inputs can be received from an EEG machine (e.g., EEG sensors 210), a heart rate monitor, a pulse oximeter, and a plurality of other ambient sensors (collectively referred to as ambient sensors 215). Configuration scripts 225 can be used to define initial input and output parameters. Active configuration scripts can be set initially based on user preference for output and based on population normative brain signals and corresponding biomarkers. In some embodiments, the system 200 can be implemented to iteratively refine configuration parameters for one or more output devices 235 based on instantaneous brain signals such that the neuromodulating audio signal can be phase-locked with the endogenous signals of the user. Configuration parameters can be refined over the course of a single session, or over the course of a plurality of sessions.

The system 200 can be implemented using any one or more of a variety of processing modules 205. In some implementations, processing module 205 can be configured to determine when and how to output a neuromodulating audio signal, compute and track endogenous signals of a user, and/or compute and track various transformations of the received data, including, but not limited to, endpoint corrected Hilbert transforms (ecHT) of the EEG signal of the user. In some embodiments, a different processor can be configured to generate the audio and drive a closed-loop playback system. Processing modules 205, or in some embodiments, a memory 230, can be used to log sensor data.

Wherever located, the electrodes and additional sensors (if any) transmit collected data to a processing module 205, which can be incorporated into the CPU. The processing module 205 can be configured to receive data from the electrodes and any sensors (e.g., EEG sensors 210, ambient sensors 215), process and/or analyze the endogenous electrical signals of the user and other data received from the sensors, and to transmit output instructions to the output device(s). In some embodiments, processing and/or analyzing the endogenous electrical signals of the user includes more than passive observation, and can include actively monitoring or tracking biological signals or biomarkers associated with the received data. The processing module can include one or more processors, ASICs, PLCs, or logic circuits.

Additional details on processing and analyzing endogenous electrical signals of the user as well as neuromodulation can be found in U.S. Pat. No. 11,166,632, U.S. Application Publication No. 2022/0225920, and Patent Cooperation Treat (PCT) Application No. PCT/US2023/084108, the entire contents of each of which is hereby incorporated by reference.

### Acoustic Neuromodulation Methods

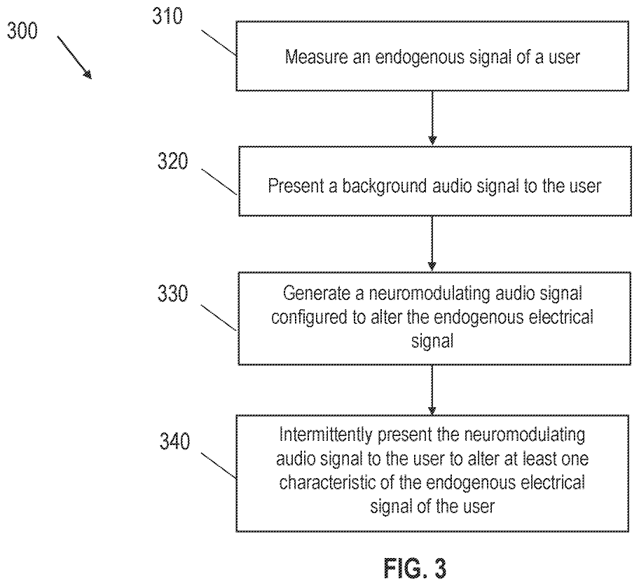

FIG. 3 illustrates an exemplary method 300 for altering an endogenous electrical signal of a user using a neuromodulating audio signal. The method 300 can be implemented by any suitable system, including but not limited to the system 200 shown and described with respect to FIG. 2. At block 310, the system implementing the method 300 can measure the endogenous electrical signal of the user. This can be performed, for example, using one or more EEG electrodes, as described above.

At block 320, the system can present a first (e.g., background) audio signal to the user. In some embodiments, the audio signal can be presented using a speaker or other audio output device incorporated into the system. As described herein, the first audio signal may not significantly alter the endogenous electrical signal. Rather, the first audio signal may be presented as a background audio signal agnostic to, or independent of, the phase of the endogenous electrical signal.

At block 330, the system can generate a second (e.g., neuromodulating) audio signal configured to alter the endogenous electrical signal. In some embodiments, generating the second audio signal can include modifying the first (e.g., background) audio signal to produce the second (e.g., neuromodulating) audio signal. The second audio signal can be configured to alter a characteristic, such as a wavelength, phase, and/or amplitude, of the endogenous electrical signal. At block 340, the system can intermittently present the second audio signal to alter at least one characteristic of the endogenous electrical signal of the user. For example, the second (e.g., neuromodulating) audio signal can alter the wavelength, phase, and/or amplitude of the endogenous electrical signal. In some embodiments, presentation of the second audio signal can occur during presentation of the first (e.g., background) audio signal, such that the user receives both the first and second audio signals simultaneously or near-simultaneously. In this manner, the first (e.g., background) audio signal may mask or disguise the second (e.g., neuromodulating) audio signal, as described herein.

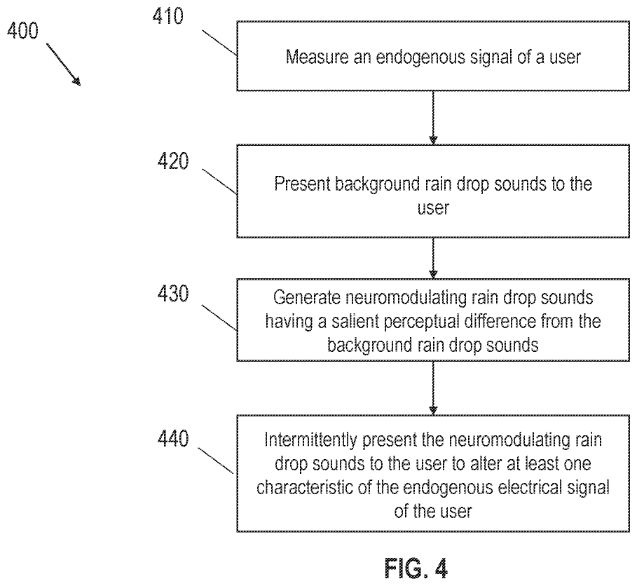

FIG. 4 illustrates an exemplary method 400 for altering an endogenous electrical signal of a user using a rain drop sounds. The method 400 can be implemented by any suitable system, including but not limited to the system 200 shown and described with respect to FIG. 2. At block 410, the system implementing the method 400 can measure the endogenous electrical signal of the user. This can be performed, for example, using one or more EEG electrodes, as described above.

At block 420, the system can present first (e.g., background) rain drop sounds to the user. In some embodiments, the rain drop sounds can be presented using a speaker or other audio output device incorporated into the system. As described herein, the first rain drop sounds may not significantly alter the endogenous electrical signal. At block 430, the system can generate second (e.g., neuromodulating) rain drop sounds. The second rain drop sounds can be configured to alter a characteristic, such as a wavelength, phase, and/or amplitude, of the endogenous electrical signal. The second rain drop sounds can have a salient perceptual difference as compared to the first rain drop sounds presented at block 420. At block 430, the system can intermittently present the second (e.g., neuromodulating) rain drop sounds to alter at least one characteristic (e.g., the wavelength, phase, or amplitude) of the endogenous electrical signal of the user. In some embodiments, presentation of the second rain drop sounds can occurs during presentation of the first (e.g., background) rain drop sounds, such that the user receives both the first and second rain drop sounds simultaneously or near-simultaneously.

### Computing Device

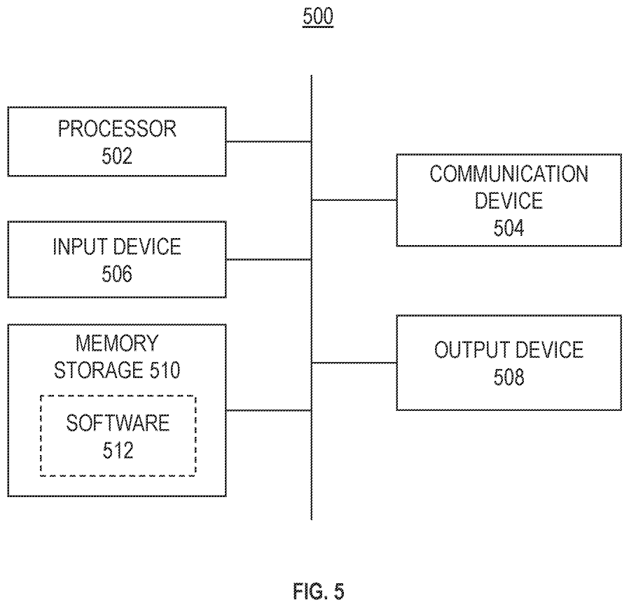

FIG. 5 depicts a computing device 500, according to one or more examples of the disclosure. In one or more examples, device 500 may be understood to encompass one or more components of the neuromodulation system 200 illustrated in FIG. 2. In one or more examples, computing device 500 may be configured to implement a method for inducing sleep using neuromodulation, such as the methods 300, 400 of FIG. 3 and FIG. 4, respectively.

Device 500 can be a host computer connected to a network. Device 500 can be a client computer or a server. As shown in FIG. 5, device 500 can be any suitable type of microprocessor-based device, such as a personal computer, workstation, server, or handheld computing device (portable electronic device) such as a phone or tablet. The device can include, for example, one or more of processors 502, input device 506, output device 508, storage 510, and communication device 504. Input device 506 and output device 508 can generally correspond to those described above and can either be connectable or integrated with the computer. For example, the one or more processors 502 may comprise processors 205. The input device 506 may comprise the one or more sensors (e.g., EEG sensors 210 and/or ambient sensors 215 described herein with respect at least to system 200 and FIG. 2). The output device 508 may comprise the output device 235. The storage 510 may comprise at least the memory 230 described herein with respect to system 200 illustrated in FIG. 2.

Input device 506 can be any suitable device that provides an input, such as a touch screen, keyboard or keypad, mouse, or voice-recognition device. Output device 508 can be any suitable device that provides output, such as a touch screen, haptics device, or speaker.

Storage 510 can be any suitable device that provides storage, such as an electrical, magnetic, or optical memory, including a RAM, cache, hard drive, or removable storage disk. Communication device 504 can include any suitable device capable of transmitting and receiving signals over a network, such as a network interface chip or device. The components of the computer can be connected in any suitable manner, such as via a physical bus or wirelessly.

Software 512, which can be stored in storage 510 and executed by processor 502, can include, for example, the programming that embodies the functionality of the present disclosure (e.g., as embodied in the devices as described above).

Software 512 can also be stored and/or transported within any non-transitory computer-readable storage medium for use by or in connection with an instruction execution system, apparatus, or device, such as those described above, that can fetch instructions associated with the software from the instruction execution system, apparatus, or device and execute the instructions. In the context of this disclosure, a computer-readable storage medium can be any medium, such as storage 510, that can contain or store programming for use by or in connection with an instruction execution system, apparatus, or device.

Software 512 can also be propagated within any transport medium for use by or in connection with an instruction execution system, apparatus, or device, such as those described above, that can fetch instructions associated with the software from the instruction execution system, apparatus, or device and execute the instructions. In the context of this disclosure, a transport medium can be any medium that can communicate, propagate, or transport programming for use by or in connection with an instruction execution system, apparatus, or device. The transport readable medium can include, but is not limited to, an electronic, magnetic, optical, electromagnetic, or infrared wired or wireless propagation medium.

Device 500 may be connected to a network, which can be any suitable type of interconnected communication system. The network can implement any suitable communications protocol and can be secured by any suitable security protocol. The network can comprise network links of any suitable arrangement that can implement the transmission and reception of network signals, such as wireless network connections, T1 or T3 lines, cable networks, DSL, or telephone lines.

Device 500 can implement any operating system suitable for operating on the network. Software 512 can be written in any suitable programming language, such as C, C++, Java, or Python. In various embodiments, application software embodying the functionality of the present disclosure can be deployed in different configurations, such as in a client/server arrangement or through a Web browser as a Web-based application or Web service, for example.

### Definitions & Conclusion

As used herein, the singular forms “a”, “an”, and “the” include the plural reference unless the context clearly dictates otherwise.

Reference to “about” a value or parameter herein includes (and describes) variations that are directed to that value or parameter per se. For example, description referring to “about X” includes description of “X”.

It is understood that aspects and variations of the invention described herein include “consisting” and/or “consisting essentially of” aspects and variations.

The term “significant” as it relates to altering an endogenous electrical signal, e.g., “does not significantly alter the endogenous electrical signal” generally refers to a background audio signal that can be presented to a user agnostic to the endogenous electrical signal of the user. In this manner, the background, or first, audio signal presented to the user may not be presented with the intention of altering (e.g., neuromodulating) the endogenous electrical signal. Rather, the background audio signal may be presented to mask the neuromodulating, or second, audio signal presented to the user, as described herein.

When a range of values or values is provided, it is to be understood that each intervening value between the upper and lower limit of that range, and any other stated or intervening value in that stated range, is encompassed within the scope of the present disclosure. Where the stated range includes upper or lower limits, ranges excluding either of those included limits are also included in the present disclosure.

The entire disclosure of the patents and publications referred in this application are hereby incorporated herein by reference for all purposes. To the extent that any reference incorporated by reference conflicts with the instant disclosure, the instant disclosure shall control.

The section headings used herein are for organization purposes only and are not to be construed as limiting the subject matter described. The description is presented to enable one of ordinary skill in the art to make and use the invention and is provided in the context of a patent application and its requirements. Various modifications to the described embodiments will be readily apparent to those persons skilled in the art and the generic principles herein may be applied to other embodiments. Thus, the present invention is not intended to be limited to the embodiment shown but is to be accorded the widest scope consistent with the principles and features described herein.

The figures illustrate processes according to various embodiments. In the exemplary processes, some blocks are, optionally, combined, the order of some blocks is, optionally, changed, and some blocks are, optionally, omitted. In some examples, additional steps may be performed in combination with the exemplary processes.

Accordingly, the operations as illustrated (and described in greater detail below) are exemplary by nature and, as such, should not be viewed as limiting.

Although the disclosure and examples have been fully described with reference to the accompanying figures, it is to be noted that various changes and modifications will become apparent to those skilled in the art. Such changes and modifications are to be understood as being included within the scope of the disclosure and examples as defined by the claims.

### EXEMPLARY EMBODIMENTS

The following embodiments are exemplary and are not intended to limit the scope of any invention described herein.

Embodiment 1. A method, comprising the steps of:
measuring an endogenous electrical signal of a user;
presenting a first audio signal to the user that does not significantly alter a wavelength, phase, or amplitude of the endogenous electrical signal;
modifying the first audio signal to produce a second, neuromodulating audio signal that is configured to alter a wavelength, phase, or amplitude of the endogenous electrical signal in a predetermined manner; and
intermittently presenting the second, neuromodulating audio signal to the user to alter the wavelength, phase, or amplitude of the endogenous electrical signal in a predetermined manner.

Embodiment 2. A method, comprising the steps of:
measuring an endogenous electrical signal of a user;
presenting a first audio signal to the user that does not significantly alter a wavelength, phase, or amplitude of the endogenous electrical signal;
generating a second, neuromodulating audio signal that is configured to alter a wavelength, phase, or amplitude of the endogenous electrical signal in a predetermined manner; and
intermittently presenting the second, neuromodulating audio signal to the user to alter the wavelength, phase, or amplitude of the endogenous electrical signal in a predetermined manner.

Embodiment 3. The method according to any preceding embodiment, wherein the first audio signal and the second audio signal comprise or consist of sounds simulating the same type of sound-producing event.

Embodiment 4. The method according to embodiment 3, wherein the sound-producing event occurs in nature.

Embodiment 5. The method according to embodiment 3, wherein the sound-producing event does not occur in nature.

Embodiment 6. The method according to any one of embodiments 1-4:
wherein the step of presenting the first audio signal to the user comprises presenting a first audio signal to a user comprising or consisting of first rain drop sounds; and
wherein the step of generating the second, neuromodulating audio signal comprises generating second rain drop sounds, the second rain drop sounds having a salient perceptual difference from the first rain drop sounds; and
wherein the step of intermittently presenting the second, neuromodulating audio signal to the user comprises presenting the second rain drop sounds to alter the wavelength, phase, or amplitude of the endogenous electrical signal in a predetermined manner.

Embodiment 7. The method according to any one of embodiments 1-4:
wherein the step of presenting the first audio signal to the user comprises presenting a first audio signal to a user comprising or consisting of sounds of a first flow of water; and
wherein the step of generating the second, neuromodulating audio signal comprises generating sounds of a second flow of water, the sounds of the second flow of water having a salient perceptual difference from the sounds of the first flow of water; and
wherein the step of intermittently presenting the second, neuromodulating audio signal to the user comprises presenting the sounds of the second flow of water to alter the wavelength, phase, or amplitude of the endogenous electrical signal in a predetermined manner.

Embodiment 8. The method according to any one of embodiments 1-4:
wherein the step of presenting the first audio signal to the user comprises presenting a first audio signal to a user comprising or consisting of first sounds of a bubbling brook; and
wherein the step of generating the second, neuromodulating audio signal comprises generating second sounds of a bubbling brook, the second sounds of the bubbling brook having a salient perceptual difference from the first sounds of the bubbling brook; and
wherein the step of intermittently presenting the second, neuromodulating audio signal to the user comprises presenting the second sounds of the bubbling brook to alter the wavelength, phase, or amplitude of the endogenous electrical signal in a predetermined manner.

Embodiment 9. The method according to any one of embodiments 1-4:
wherein the step of presenting the first audio signal to the user comprises presenting a first audio signal to a user comprising or consisting of first forest sounds; and
wherein the step of generating the second, neuromodulating audio signal comprises generating second forest sounds, the second forest sounds having a salient perceptual difference from the first forest sounds; and
wherein the step of intermittently presenting the second, neuromodulating audio signal to the user comprises presenting the second forest sounds to alter the wavelength, phase, or amplitude of the endogenous electrical signal in a predetermined manner.

Embodiment 10. The method according to any one of embodiments 1-4:
wherein the step of presenting the first audio signal to the user comprises presenting a first audio signal to a user comprising or consisting of sounds simulating a first type of sound-producing event selected from the group consisting of: rain drop sounds, sounds of a flow of water, sounds of a bubbling brook, forest sounds, leaf sounds, record player sounds, sounds of children playing, thunderstorm sounds, ocean sounds, wildlife sounds, and logs crackling in a fire;
wherein the step of generating the second audio signal comprises generating sounds simulating a second type of sound-producing event selected from the group consisting of: rain drop sounds, sounds of a flow of water, sounds of a bubbling brook, forest sounds, leaf sounds, record player sounds, sounds of children playing, thunderstorm sounds, ocean sounds, wildlife sounds, and logs crackling in a fire;
wherein the sounds simulating the second type of sound-producing event have a salient perceptual difference from the sounds simulating the first sound-producing event;
wherein the step of intermittently presenting the second, neuromodulating audio signal to the user comprises presenting the sounds simulating the second sound-producing event to alter the wavelength, phase, or amplitude of the endogenous electrical signal in a predetermined manner.

Embodiment 11. The method according to any one of embodiments 1-3 or 5:
wherein the step of presenting the first audio signal to the user comprises presenting a first audio signal to a user comprising or consisting of first synthesized sounds; and
wherein the step of generating the second, neuromodulating audio signal comprises generating second synthesized sounds, the second synthesized sounds having a salient perceptual difference from the first synthesized sounds; and
wherein the step of intermittently presenting the second, neuromodulating audio signal to the user comprises presenting the second synthesized sounds.

Embodiment 12. The method according to any one of embodiments 1-4, wherein the first audio signal comprises or consists of first rain drop sounds and the second, neuromodulating audio signal comprises or consists of second rain drop sounds, wherein the second raindrop sounds have a salient perceptual difference from the first rain drop sounds.

Embodiment 13. The method according to any one of embodiments 1-4, wherein the first audio signal comprises or consists of sounds of a first flow of water and the second, neuromodulating audio signal comprises or consists of sounds of a second flow of water, wherein the sounds of the second flow of water have a salient perceptual difference from the sounds of the first flow of water.

Embodiment 14. The method according to any one of embodiments 1-4, wherein the first audio signal comprises or consists of first sounds of a bubbling brook and the second, neuromodulating audio signal comprises or consists of second sounds of a bubbling brook, wherein the second sounds of the bubbling brook have a salient perceptual difference from the sounds of the first sounds of the bubbling brook.

Embodiment 15. The method according to any one of embodiments 1-4, wherein the first audio signal comprises or consists of first forest sounds and the second, neuromodulating audio signal comprises or consists of second forest sounds, wherein the second forest sounds have a salient perceptual difference from the first forest sounds.

Embodiment 16. The method according to any one of embodiments 1-4,
wherein the first audio signal comprises or consists of sounds simulating a first sound-producing event selected from the group consisting of: rain drop sounds, sounds of a flow of water, sounds of a bubbling brook, forest sounds, leaf sounds, record player sounds, sounds of children playing, thunderstorm sounds, ocean sounds, wildlife sounds, and logs crackling in a fire;

wherein the second, neuromodulating audio signal comprises or consists of sounds simulating a second sound-producing event selected from the group consisting of: rain drop sounds, sounds of a flow of water, sounds of a bubbling brook, forest sounds, leaf sounds, record player sounds, sounds of children playing, thunderstorm sounds, ocean sounds, wildlife sounds, and logs crackling in a fire; and
wherein the sounds simulating the second, neuromodulating sound-producing event have a salient perceptual difference from the sounds simulating the first sound-producing event.

Embodiment 17. The method according to any one of the foregoing embodiments, wherein the first audio signal comprises or consists of first synthesized sounds and the second, neuromodulating audio signal comprises or consists of second synthesized sounds, wherein the second synthesized sounds have a salient perceptual difference from the first synthesized sounds.

Embodiment 18. The method according to any one of the foregoing embodiments, wherein a sound pressure level (SPL) calculated from a root mean square (RMS) energy of the second, neuromodulating audio signal is at least 4 dB, at least 5 dB, at least 10 dB, at least 15 dB, at least 20 dB or even higher than the SPL calculated from an RMS energy of the first audio signal.

Embodiment 19. The method according to any one of the foregoing embodiments, wherein the SPL of the first audio signal is from about 23 dB to about 60 dB, from about 23 dB to about 50 dB, from about 30 dB to about 60 dB, from about 30 dB to about 50 dB, from about 40 dB to about 60 dB, or from about 40 dB to about 40 dB, including any and all ranges and sub-ranges therein.

Embodiment 20. The method according to any one of the foregoing embodiments, wherein the SPL of the second, neuromodulating signal is from about 50 dB to about 85 dB, from about 50 dB to about 75 dB, from about 60 dB to about 85 dB, or from about 60 dB to about 75 dB, including any and all ranges and sub-ranges therein.

Embodiment 21. A method for acoustic neuromodulation, comprising:
measuring an endogenous electrical signal of a user;
presenting a background audio signal to the user;
based on the measured endogenous electrical signal, generating a neuromodulating audio signal configured to alter the endogenous electrical signal; and
intermittently presenting the neuromodulating audio signal to the user to alter at least one characteristic of the endogenous electrical signal.

Embodiment 22. The method of embodiment 21, wherein generating the neuromodulating audio signal comprises modifying the background audio signal to generate the neuromodulating audio signal.

Embodiment 23. The method of embodiment 21 or 22, wherein intermittently presenting the neuromodulating audio signal occurs while simultaneously continuously presenting the background audio signal.

Embodiment 24. The method of any one of embodiments 21-23, wherein the at least one characteristic of the endogenous electrical signal comprises a wavelength, a phase, or an amplitude of the endogenous electrical signal.

Embodiment 25. The method of any one of embodiments 21-24, wherein the background audio signal and the neuromodulating audio signal comprise sounds simulating the same type of sound-producing event.

Embodiment 26. The method of embodiment 24, wherein the sound-producing event occurs in nature.

Embodiment 27. The method of any one of embodiments 21-26, wherein:
presenting the background audio signal to the user comprises presenting background rain drop sounds;
generating the neuromodulating audio signal comprises generating neuromodulating rain drop sounds having a salient perceptual difference from the background rain drop sounds; and
intermittently presenting the neuromodulating audio signal to the user comprises presenting the neuromodulating rain drop sounds to alter the at least one characteristic of the endogenous electrical signal.

Embodiment 28. The method of any one of embodiments 21-26, wherein:
presenting the background audio signal to the user comprises presenting background sounds of a flow of water;
generating the neuromodulating audio signal comprises generating neuromodulating sounds of a flow of water having a salient perceptual difference from the background sounds of the flow of water; and
intermittently presenting the neuromodulating audio signal to the user comprises presenting the neuromodulating sounds of the flow of water to alter the at least one characteristic of the endogenous electrical signal.

Embodiment 29. The method of any one of embodiments 21-26, wherein:
presenting the background audio signal to the user comprises presenting background sounds of a bubbling brook;
generating the neuromodulating audio signal comprises generating neuromodulating sounds of a bubbling brook having a salient perceptual difference from the background sounds of the bubbling brook; and
intermittently presenting the neuromodulating audio signal to the user comprises presenting the neuromodulating sounds of the bubbling brook to alter the at least one characteristic of the endogenous electrical signal.

Embodiment 30. The method of any one of embodiments 21-26, wherein:
presenting the background audio signal to the user comprises presenting background forest sounds;
of generating the neuromodulating audio signal comprises generating neuromodulating forest sounds having a salient perceptual difference from the background forest sounds; and
intermittently presenting the neuromodulating audio signal to the user comprises presenting the neuromodulating forest sounds to alter the at least one characteristic of the endogenous electrical signal.

Embodiment 31. The method of any one of embodiments 21-26, wherein:
presenting the background audio signal to the user comprises presenting background sounds simulating a sound-producing event selected from the group consisting of: rain drop sounds, sounds of a flow of water, sounds of a bubbling brook, forest sounds, leaf sounds, record player sounds, sounds of children playing, thunderstorm sounds, ocean sounds, wildlife sounds, and sounds of logs crackling in a fire;
generating the neuromodulating audio signal comprises generating neuromodulating sounds simulating a sound-producing event selected from the group consisting of: rain drop sounds, sounds of a flow of water, sounds of a bubbling brook, forest sounds, leaf sounds, record player sounds, sounds of children playing, thunderstorm sounds, ocean sounds, wildlife sounds, and logs crackling in a fire,
wherein the neuromodulating sounds simulating the sound-producing event have a salient perceptual difference from the background sounds simulating the sound-producing event, and the background sounds and the neuromodulating sounds are the same type of sound-producing event; and
intermittently presenting the neuromodulating audio signal to the user comprises presenting the neuromodulating sounds simulating the sound-producing event to alter the at least one characteristic of the endogenous electrical signal.

Embodiment 32. The method of any one of embodiments 21-31, wherein:
presenting the background audio signal to the user comprises presenting background synthesized sounds;
generating the neuromodulating audio signal comprises generating neuromodulating synthesized sounds having a salient perceptual difference from the background synthesized sounds; and
intermittently presenting the neuromodulating audio signal to the user comprises presenting the neuromodulating synthesized sounds to alter the at least one characteristic of the endogenous electrical signal.

Embodiment 33. The method of any one of embodiments 21-32, wherein a sound pressure level (SPL) calculated from a root mean square (RMS) energy of the neuromodulating audio signal is at least 4 dB, at least 5 dB, at least 10 dB, at least 15 dB, or at least 20 dB higher than an SPL calculated from an RMS energy of the background audio signal.

Embodiment 34. The method of any one of embodiments 21-33, wherein the SPL of the background audio signal is from about 23 dB to about 60 dB, from about 23 dB to about 50 dB, from about 30 dB to about 60 dB, from about 30 dB to about 50 dB, from about 40 dB to about 60 dB, or from about 40 dB to about 50 dB.

Embodiment 35. The method of any one of embodiments 21-34, wherein the SPL of the neuromodulating audio signal is from about 50 dB to about 85 dB, from about 50 dB to about 75 dB, from about 60 dB to about 85 dB, or from about 60 dB to about 75 dB.

Embodiment 36. A system for acoustic neuromodulation, comprising:
one or more sensors configured to measure an endogenous electrical signal of a user;
a processing device configured to:
   generate a background audio signal; and
   based on the measured endogenous electrical signal, generate a neuromodulating audio signal configured to alter at least one characteristic of the endogenous electrical signal; and
one or more speakers configured to:
   present the background audio signal to the user; and
   intermittently present the neuromodulating audio signal to the user to alter the at least one characteristic of the endogenous electrical signal.

Embodiment 37. The system of embodiment 36, wherein generating the neuromodulating audio signal comprises modifying the background audio signal to generate the neuromodulating audio signal.

Embodiment 38. The system of embodiment 36 or 37, wherein intermittently presenting the neuromodulating audio signal occurs while simultaneously continuously presenting the background audio signal.

Embodiment 39. The system of any one of embodiments 36-38, wherein the at least one characteristic of the endogenous electrical signal comprises a wavelength, a phase, or an amplitude of the endogenous electrical signal.

Embodiment 40. The system of any one of embodiment 36-39, wherein the background audio signal and the neuromodulating audio signal comprise sounds simulating the same type of sound-producing event.

Embodiment 41. The system of embodiment 40, wherein the sound-producing event occurs in nature.

Embodiment 42. The system of any one of embodiments 36-41, wherein:
presenting the background audio signal to the user comprises presenting background rain drop sounds;
generating the neuromodulating audio signal comprises generating neuromodulating rain drop sounds having a salient perceptual difference from the background rain drop sounds; and
intermittently presenting the neuromodulating audio signal to the user comprises presenting the neuromodulating rain drop sounds to alter the at least one characteristic of the endogenous electrical signal.

Embodiment 43. The system of any one of embodiments 36-41, wherein:
presenting the background audio signal to the user comprises presenting background sounds of a flow of water;
generating the neuromodulating audio signal comprises generating neuromodulating sounds of a flow of water having a salient perceptual difference from the background sounds of the flow of water; and
intermittently presenting the neuromodulating audio signal to the user comprises presenting the neuromodulating sounds of the flow of water to alter the at least one characteristic of the endogenous electrical signal.

Embodiment 44. The system of any one of embodiments 36-41, wherein:
presenting the background audio signal to the user comprises presenting background sounds of a bubbling brook;
generating the neuromodulating audio signal comprises generating neuromodulating sounds of a bubbling brook having a salient perceptual difference from the background sounds of the bubbling brook; and
intermittently presenting the neuromodulating audio signal to the user comprises presenting the neuromodulating sounds of the bubbling brook to alter the at least one characteristic of the endogenous electrical signal.

Embodiment 45. The system of any one of embodiments 36-41, wherein:
presenting the background audio signal to the user comprises presenting background forest sounds;
of generating the neuromodulating audio signal comprises generating neuromodulating forest sounds having a salient perceptual difference from the background forest sounds; and
intermittently presenting the neuromodulating audio signal to the user comprises presenting the neuromodulating forest sounds to alter the at least one characteristic of the endogenous electrical signal.

Embodiment 46. The system of any one of embodiments 36-41, wherein:
presenting the background audio signal to the user comprises presenting background sounds simulating a sound-producing event selected from the group consisting of: rain drop sounds, sounds of a flow of water, sounds of a bubbling brook, forest sounds, leaf sounds, record player sounds, sounds of children playing, thunderstorm sounds, ocean sounds, wildlife sounds, and sounds of logs crackling in a fire;
generating the neuromodulating audio signal comprises generating neuromodulating sounds simulating a sound-producing event selected from the group consisting of: rain drop sounds, sounds of a flow of water, sounds of a bubbling brook, forest sounds, leaf sounds, record player sounds, sounds of children playing, thunderstorm sounds, ocean sounds, wildlife sounds, and logs crackling in a fire,
wherein the neuromodulating sounds simulating the sound-producing event have a salient perceptual difference from the background sounds simulating the sound-producing event, and the background sounds and the neuromodulating sounds are the same type of sound-producing event; and
intermittently presenting the neuromodulating audio signal to the user comprises presenting the neuromodulating sounds simulating the sound-producing event to alter the at least one characteristic of the endogenous electrical signal.

Embodiment 47. The system of any one of embodiments 36-46, wherein:
presenting the background audio signal to the user comprises presenting background synthesized sounds;
generating the neuromodulating audio signal comprises generating neuromodulating synthesized sounds having a salient perceptual difference from the background synthesized sounds; and
intermittently presenting the neuromodulating audio signal to the user comprises presenting the neuromodulating synthesized sounds to alter the at least one characteristic of the endogenous electrical signal.

Embodiment 48. The system of any one of embodiments 36-47, wherein a sound pressure level (SPL) calculated from a root mean square (RMS) energy of the neuromodulating audio signal is at least 4 dB, at least 5 dB, at least 10 dB, at least 15 dB, or at least 20 dB higher than an SPL calculated from an RMS energy of the background audio signal.

Embodiment 49. The system of any one of embodiments 36-48, wherein the SPL of the background audio signal is from about 23 dB to about 60 dB, from about 23 dB to about 50 dB, from about 30 dB to about 60 dB, from about 30 dB to about 50 dB, from about 40 dB to about 60 dB, or from about 40 dB to about 50 dB.

Embodiment 50. The system of any one of embodiments 36-49, wherein the SPL of the neuromodulating audio signal is from about 50 dB to about 85 dB, from about 50 dB to about 75 dB, from about 60 dB to about 85 dB, or from about 60 dB to about 75 dB.

Embodiment 51. A non-transitory computer-readable storage medium storing instructions that, when executed by a processing device operatively coupled to one or more sensors configured to measure an endogenous electrical signal of a user and to one or more speakers configured to present audio signals, cause the device to:
generate a background audio signal;
based on the measured endogenous electrical signal, generate a neuromodulating audio signal configured to alter at least one characteristic of the endogenous electrical signal; and
cause the one or more speakers to:
   present the background audio signal to the user; and
   intermittently present the neuromodulating audio signal to the user to alter the at least one characteristic of the endogenous electrical signal.

1. A method for acoustic neuromodulation, comprising:
measuring an endogenous electrical signal of a user;
presenting a background audio signal to the user;
based on the measured endogenous electrical signal, generating a neuromodulating audio signal configured to alter the endogenous electrical signal; and
intermittently presenting the neuromodulating audio signal to the user to alter at least one characteristic of the endogenous electrical signal.

2. The method of claim 1, wherein generating the neuromodulating audio signal comprises modifying the background audio signal to generate the neuromodulating audio signal.

3. The method of claim 1, wherein intermittently presenting the neuromodulating audio signal occurs while simultaneously continuously presenting the background audio signal.

4. The method of claim 1, wherein the at least one characteristic of the endogenous electrical signal comprises a wavelength, a phase, or an amplitude of the endogenous electrical signal.

5. The method claim 1, wherein the background audio signal and the neuromodulating audio signal comprise sounds simulating the same type of sound-producing event.

6. The method of claim 5, wherein the sound-producing event occurs in nature.

7-10. (canceled)

11. The method of claim 1, wherein:
presenting the background audio signal to the user comprises presenting background sounds simulating a sound-producing event selected from the group consisting of: rain drop sounds, sounds of a flow of water, sounds of a bubbling brook, forest sounds, leaf sounds, record player sounds, sounds of children playing, thunderstorm sounds, ocean sounds, wildlife sounds, and sounds of logs crackling in a fire;
generating the neuromodulating audio signal comprises generating neuromodulating sounds simulating a sound-producing event selected from the group consisting of: rain drop sounds, sounds of a flow of water, sounds of a bubbling brook, forest sounds, leaf sounds, record player sounds, sounds of children playing, thunderstorm sounds, ocean sounds, wildlife sounds, and logs crackling in a fire,
wherein the neuromodulating sounds simulating the sound-producing event have a salient perceptual difference from the background sounds simulating the sound-producing event, and the background sounds and the neuromodulating sounds are the same type of sound-producing event; and
intermittently presenting the neuromodulating audio signal to the user comprises presenting the neuromodulating sounds simulating the sound-producing event to alter the at least one characteristic of the endogenous electrical signal.

12. The method of claim 1, wherein:
presenting the background audio signal to the user comprises presenting background synthesized sounds;
generating the neuromodulating audio signal comprises generating neuromodulating synthesized sounds having a salient perceptual difference from the background synthesized sounds; and
intermittently presenting the neuromodulating audio signal to the user comprises presenting the neuromodulating synthesized sounds to alter the at least one characteristic of the endogenous electrical signal.

13. The method of claim 1, wherein a sound pressure level (SPL) calculated from a root mean square (RMS) energy of the neuromodulating audio signal is at least 4 dB, higher than an SPL calculated from an RMS energy of the background audio signal.

14. The method of claim 1, wherein the SPL of the background audio signal is from about 23 dB to about 60 dB.

15. The method of claim 1, wherein the SPL of the neuromodulating audio signal is from about 50 dB to about 85 dB.

16. A system for acoustic neuromodulation, comprising:
one or more sensors configured to measure an endogenous electrical signal of a user;
a processing device configured to:
generate a background audio signal; and
based on the measured endogenous electrical signal, generate a neuromodulating audio signal configured to alter at least one characteristic of the endogenous electrical signal; and
one or more speakers configured to:
present the background audio signal to the user; and
intermittently present the neuromodulating audio signal to the user to alter the at least one characteristic of the endogenous electrical signal.

17. The system of claim 16, wherein generating the neuromodulating audio signal comprises modifying the background audio signal to generate the neuromodulating audio signal.

18. The system of claim 16, wherein intermittently presenting the neuromodulating audio signal occurs while simultaneously continuously presenting the background audio signal.

19. The system of claim 16, wherein the at least one characteristic of the endogenous electrical signal comprises a wavelength, a phase, or an amplitude of the endogenous electrical signal.

20. The system of claim 16, wherein the background audio signal and the neuromodulating audio signal comprise sounds simulating the same type of sound-producing event.

21-27. (canceled)

28. The system of claim 16, wherein a sound pressure level (SPL) calculated from a root mean square (RMS) energy of the neuromodulating audio signal is at least 4 dB higher than an SPL calculated from an RMS energy of the background audio signal.

29. The system of claim 16, wherein the SPL of the background audio signal is from about 23 dB to about 60 dB.

30. The system of claim 16, wherein the SPL of the neuromodulating audio signal is from about 50 dB to about 85 dB.

31. A non-transitory computer-readable storage medium storing instructions that, when executed by a processing device operatively coupled to one or more sensors configured to measure an endogenous electrical signal of a user and to one or more speakers configured to present audio signals, cause the device to:
generate a background audio signal;
based on the measured endogenous electrical signal, generate a neuromodulating audio signal configured to alter at least one characteristic of the endogenous electrical signal; and
cause the one or more speakers to:
present the background audio signal to the user; and
intermittently present the neuromodulating audio signal to the user to alter the at least one characteristic of the endogenous electrical signal.
